(19) United States
(12) Patent Application Publication
(10) Pub. No.: US 2019/0311798 A1
(43) Pub. Date: Oct. 10, 2019

(54) Computing Devices with Improved User Interfaces for Applications

(71) Applicant: Sutter Health, Emeryville, CA (US)

(72) Inventors: Scott D. Imahara, Scotts Valley, CA (US); Essex Powell, Capitola, CA (US); Holly Nath, Los Altos, CA (US)

(21) Appl. No.: 16/276,507

(22) Filed: Feb. 14, 2019

Related U.S. Application Data
(60) Provisional application No. 62/655,658, filed on Apr. 10, 2018.

(51) Int. Cl. G16H 40/20; G06Q 10/06; G06F 3/0481; G06F 3/0482

(52) U.S. Cl. CPC G16H 40/20; G06F 3/0482; G06F 3/04817; G06Q 10/06393

(57) ABSTRACT

Exemplary embodiments include a computing device configured to display on a screen a menu listing one or more applications, and additionally being configured to display on the screen an application summary that can be reached directly from the menu, wherein the application summary displays a limited list of data offered within the one or more applications. Further, the application summary may be displayed while one or more other applications are not displayed.

### CROSS REFERENCE TO RELATED APPLICATIONS

This application claims the priority benefit of U.S. Provisional Patent Application Ser. No. 62/655,658 filed on Apr. 10, 2018 and titled “Healthcare Provider Dashboards,” which is hereby incorporated by reference in its entirety.

### FIELD OF THE TECHNOLOGY

Embodiments of the disclosure relate to computing devices with improved user interfaces for applications.

### SUMMARY

Provided herein are exemplary systems including a computing device comprising a display screen, the computing device being configured to dynamically display a specific, structured interactive graphical user interface paired with a prescribed functionality directly related to the interactive graphical user interface's structure. Further embodiments include the structure dynamically displaying a limited set of information to a user, with the prescribed functionality utilizing a N2S methodology. According to various exemplary embodiments, the N2S methodology causes transformation of a message into a specifically dynamic visually perceptible graphical icon. The dynamic visually perceptible graphical icon may represent a condensed information set.

In certain exemplary embodiments, the condensed information set may be dynamically displayed along with another condensed information set on a limited screen size. The limited screen size may be on a mobile computing device, and the transformation may increase a speed of the display. Further, the transformation may increase an amount of information dynamically displayed on a limited screen size or on a display of any type of computing device.

The computing device, according to many exemplary embodiments, may be configured to display on the screen a menu listing one or more applications, and additionally being configured to display on the screen an application summary that can be reached directly from the menu, wherein the application summary displays a limited list of data offered within the one or more applications. Further, the application summary may be displayed while one or more other applications are not displayed.

In various exemplary embodiments, the computing device is configured to display on the screen a menu listing one or more applications, and additionally being configured to display on the screen an indicator that can be reached directly from the menu, wherein the indicator displays a limited data set offered within the one or more applications. An application launcher may list names and/or icons of several applications and by selecting a name or icon causes a summary for that application to be opened. A kind of content of a summary for a given application may be selected using a process in which the device learns what data type is of interest to any given user. In some embodiments, a user can define what data type is of interest to that user for the summary for an application. In some embodiments, the data type for a summary for a given application varies with the environment of the device. In other embodiments, the data type for a summary for a given application varies with the actions of a user.

The summary, in various exemplary embodiments, is a frame which includes the name of one or more applications. The summary may further display a limited list of functions offered in the one or more applications. In some embodiments, the computing device is a personal computer (“PC”), and it may display some or all information in a fashion that complies with Health Insurance Portability and Accountability Act. In further embodiments, a displayed icon may represent a thumbs-up symbol with a numerical indicator between a thumb and index finger. The numerical indicator may represent a performance standard, and the performance standard may be based on N2S methodology.

In some exemplary embodiments, a data type for a summary for a given application varies with the environment of the device and dynamically displays some or all information in a fashion that complies with Health Insurance Portability and Accountability Act as appropriate for the environment of the device.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

FIG. 1 illustrates an exemplary Number to See (“N2S”) metric.

FIG. 2 illustrates an exemplary DocDays (“DD”) metric interface.

FIG. 3 shows an exemplary improved user interface for enhanced healthcare patient access and treatment.

FIG. 4 shows another exemplary improved user interface for enhanced healthcare patient access and treatment.

FIGS. 5-6 show various other exemplary improved user interfaces for enhanced healthcare patient access and treatment.

### DETAILED DESCRIPTION

In the following description, for purposes of explanation, numerous specific details are set forth in order to provide a thorough understanding of the disclosure. It will be apparent, however, to one skilled in the art, that the disclosure may be practiced without these specific details. In other instances, structures and devices are shown in block diagram form only in order to avoid obscuring the disclosure.

Healthcare providers comprise a wide variety of providers, including “specialty providers” or “specialists” or “providers.” Their practices are characterized in part by the unpredictable patient demand that they encounter, often in part because they are called upon to solve a specific patient problem due to their unique and focused expertise. In general, once specialty care is provided, the patient moves on to their routine general practice provider who typically has a fixed number of patients and a more predictable patient demand. Although the various exemplary embodiments described and illustrated herein are capable of solving the needs of many types of healthcare providers, they are especially well-suited for the “specialty providers” or “specialists” or “providers” as characterized above.

Ensuring that new patients have prompt outpatient access to specialists within a multispecialty group, is often of high priority both to patients and to a healthcare organization. Poor outpatient access often results in several negative outcomes including poor patient satisfaction, worsening patient morbidity, and patient attrition to other health care organizations.

Facilitating prompt access to specialists requires balancing patient demand and specialist supply. Imbalances in demand and supply result in multiple negative outcomes, most notably the following: 1) if patient demand exceeds provider supply, then poor access results; whereas, 2) if provider supply exceeds patient demand, then unfilled openings in the providers' schedules (i.e., “White Space”) will result, which is a waste of physician and organizational resources.

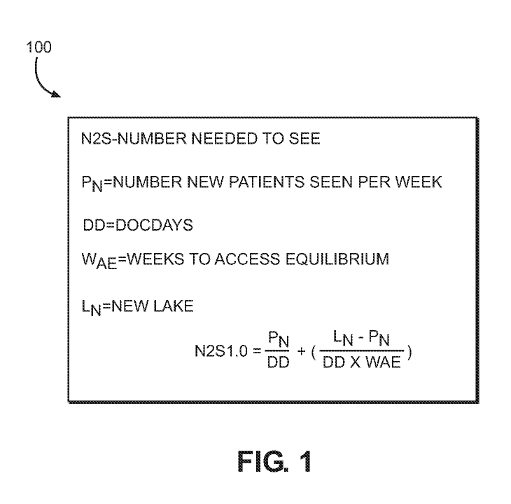

FIG. 1 illustrates an exemplary N2S metric 100. According to various exemplary embodiments, the improved user interfaces for enhanced healthcare patient access and treatment utilize a Number to See (“N2S”) metric. N2S is a formula that generates three results:

1) The “Current State”: it produces a metric for the current number of new patients each healthcare provider is seeing on average per full day of clinic (defined as 8 hours).

2) “Future State” or goal: the N2S formula produces a clear, actionable metric for the future state/goal of how many new patients a provider needs to see on average per full day of clinic to reach access equilibrium.

3) The “Gap”: the N2S formula determines how many additional new patients each provider needs to see per day to reach access equilibrium.

### Background Terms

The “N2S equation” determines the total number of new patients a specialty provider or group of providers need(s) to see per day to reach access equilibrium.

“Access equilibrium” is defined as a state where provider supply and patient demand are in balance, or a state where access is prompt for patients.

“Access ecosystem” is defined as the term to describe the environment in which a provider or group of providers deliver health care which is subject to its own unique provider supply and patient demand.

“Specialty care” is defined as the access ecosystem for specialists. It is primarily dictated by patient demand for problem-based care, and thus demand can be measured in terms of a “new lake” or backlog. The greater the “new lake” or backlog for specialists, the poorer the access. This is distinguished from primary care (e.g., internal medicine, family medicine, or pediatrics) whose providers care for a fixed population of patients or panel, by which demand is not driven by a “new lake” or backlog of patients. The N2S metric models specifically address the access ecosystem in specialty care.

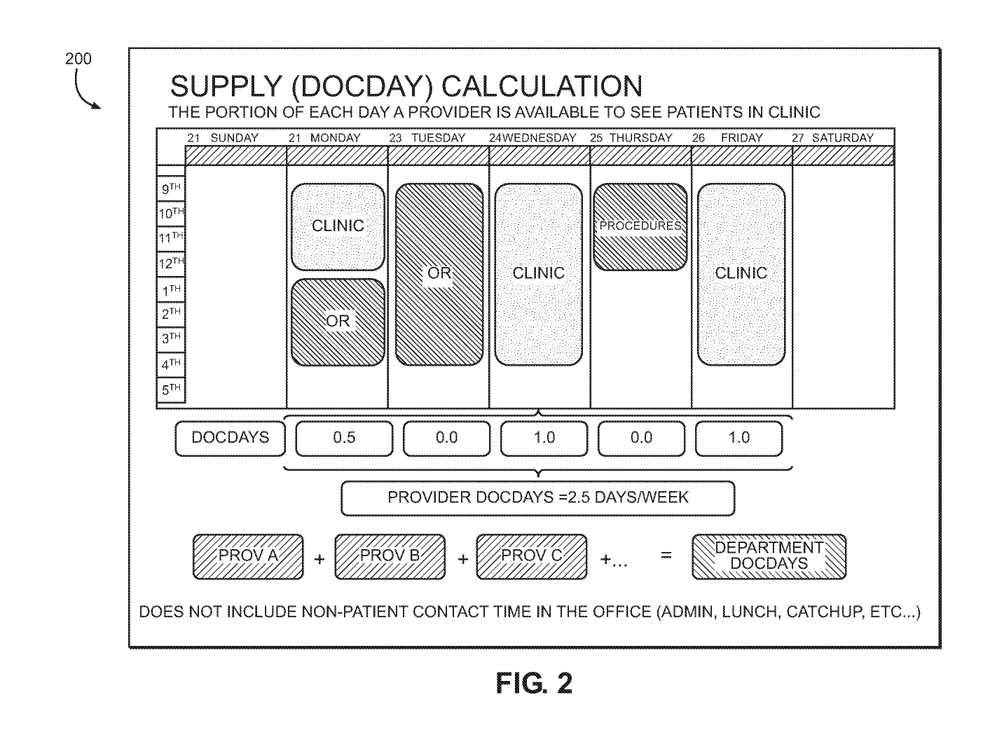

“Provider supply” is defined as the term used in specialty access to measure the amount of provider time that is available on a provider level or for a group of providers to care for patients in the clinic. “DocDays” or (DD) is the unique metric developed to measure provider supply. FIG. 2 illustrates an exemplary DD metric interface. According to various exemplary embodiments, the improved user interfaces for enhanced healthcare patient access and treatment utilize a DD metric.

“Patient demand” is the term used in specialty access to estimate the number of patients requesting care from a provider or group of providers.

“New patients” are defined in concordance with the American Medical Association's definition, to mean a patient who has not received any professional services, i.e., evaluation and management service or other face-to-face service (e.g., surgical procedure) from a physician or physician group practice (same physician specialty) within the previous three years.

Referring to FIG. 1, the N2S components include:

i) DocDays (DD): a unique metric for provider supply termed “DocDays,” which is equal to the number of days per week a provider is available to see patients.

ii) Number of new patients seen per week (“P_{N}”): defined as the number of new patients seen by a provider or specialty department or group of providers per week in the clinic.

iii) New Lake (“L_{N}”): a term used to define the number of new patients scheduled to see a provider from now and into the future, from time point zero to the maximal date in the future that patients can be scheduled.

iv) Weeks to access equilibrium (“W_{AE}”): a numerical term used to modify the N2S number. W_{AE} is a numerical value of time in weeks which clarify how long it will take providers or groups of providers to reach access equilibrium if the N2S goal (number of new patients needed to see) is met each day.

The relationships of the N2S elements to each other in FIG. 1 include:

i) N2S: accounts for the relationship of provider supply to patient demand.

ii) The “left side” of the equation (P_{N}/DD) determines the current number of new patients seen by a provider or a group of providers per day in the clinic.

iii) The “right side” of the equation, termed the “Lake Modifier” or the “Gap”, [(L_{N}−P_{N})/[DD×W_{AE})] determines the additional number of new patients needed per day to reach access equilibrium over the time period W_{AE}. The numerator of this component of the N2S equation ((L_{N}−P_{N})), is termed the “lake residual,” and establishes the number of patients still remaining in a provider's or a group of providers' schedule(s) at the conclusion of each week.

iv) The N2S equation establishes that the greater the lake residual, the greater the number of patients waiting to see a given provider or a group of providers, and thus the poorer the timely access is to that specialist.

v) Given each provider or group of providers have their own unique provider supply (DocDays) and unique patient demand (size of the new lake), existing in their own unique access ecosystem, then each provider or group of providers has their own unique N2S number or goal.

Referring to FIG. 2, an exemplary DocDay metric interface 200 is shown. A DocDay is the proportion of a day that a provider is available to see patients in the clinic. Also shown in FIG. 2 is the summation of DocDays for multiple providers for a particular department.

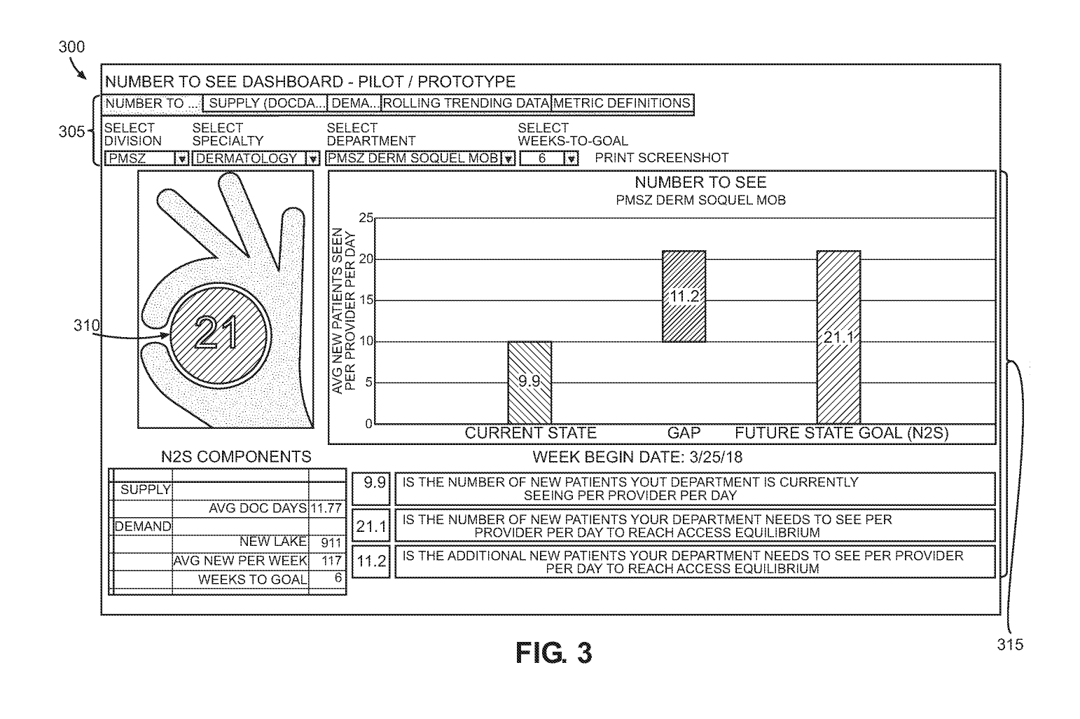

FIG. 3 shows an exemplary improved user interface 300 for enhanced healthcare patient access and treatment. Also shown in FIG. 3 are menu selections 305, icon 310 and condensed information set 315.

In most embodiments, a computing device includes a display screen to dynamically display a specific, structured interactive graphical user interface, such as interface 300 paired with a prescribed functionality directly related to the interactive graphical user interface's structure.

As shown in FIG. 3, the structure dynamically displays a limited set of information to a user. In many exemplary embodiments, the prescribed functionality utilizes the N2S methodology as described above and illustrated in FIG. 1. The N2S methodology causes transformation of a message (typically comprising one or more elements of the N2S methodology) into a specifically dynamic visually perceptible graphical icon, such as icon 310. The dynamic visually perceptible graphical icon represents a condensed information set. In many exemplary embodiments, a condensed information set may be dynamically displayed along with another condensed information set, such as condensed information set 315 on a limited screen size. The limited screen size may be on a mobile computing device, a personal computer or any other form of computing device. In most exemplary embodiments, the transformation causes an increase in the speed of the display and increases the amount of information dynamically displayed on a limited screen size.

Also shown in item 310 of FIG. 3 is the N2S number of 21 in the image or indicator of an “OK” sign. 21 represents the total number of new patients a specialist or group of specialists need(s) to see per day to reach access equilibrium. The “OK” symbol is used to represent the N2S metric, and is always shown even when the value is unacceptable. In some embodiments, the acceptable N2S number or range may be predetermined and associated with a corresponding indicator of acceptability or performance. Also shown in FIG. 3 is the number of new patients a department is currently seeing per provider day, otherwise known as the current state. In this example, the current state is 9.9. Additionally, shown is the number of additional new patients a department needs to see per provider per day to reach access equilibrium, otherwise known as the Gap. Here, the Gap is 11.2. Also illustrated to the left in FIG. 3 are the components of the N2S determination.

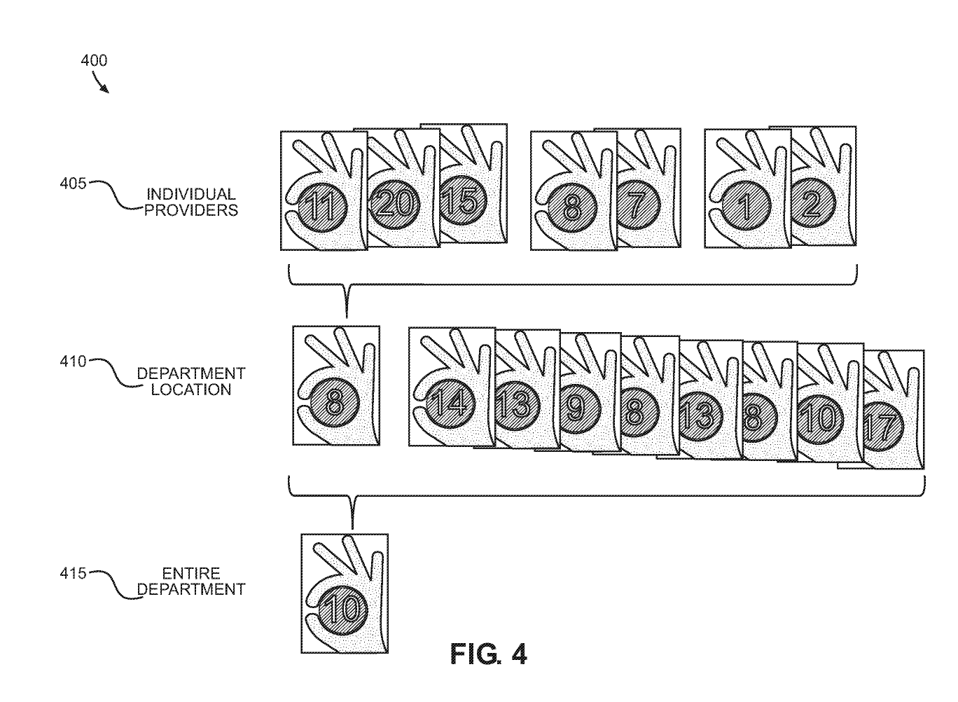

FIG. 4 shows another exemplary improved user interface 400 for enhanced healthcare patient access and treatment.

As illustrated in FIG. 4, the N2S numbers and associated indicators are shown for individual providers 405, department locations 410 and for an entire department 415. As shown and described in connection with FIG. 3, the N2S methodology causes transformation of a message (typically comprising one or more elements of the N2S methodology) into specifically dynamic visually perceptible graphical icons. Together, the dynamic visually perceptible graphical icons represents a condensed information set.

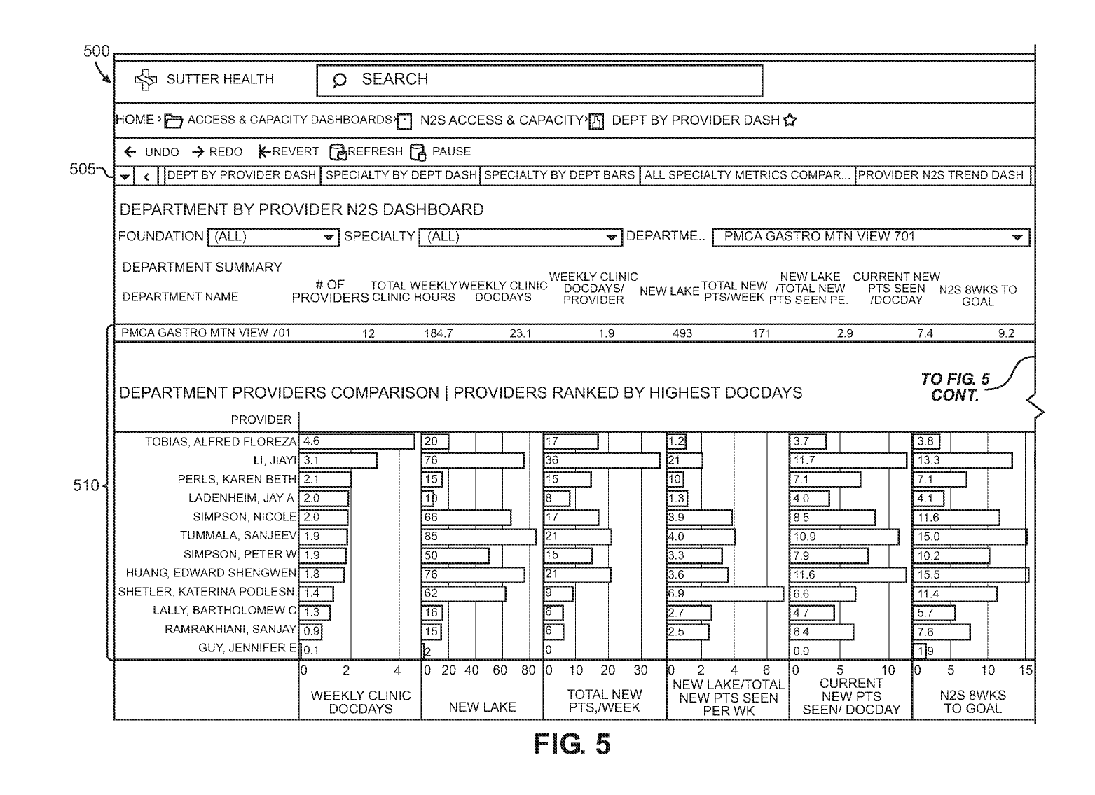

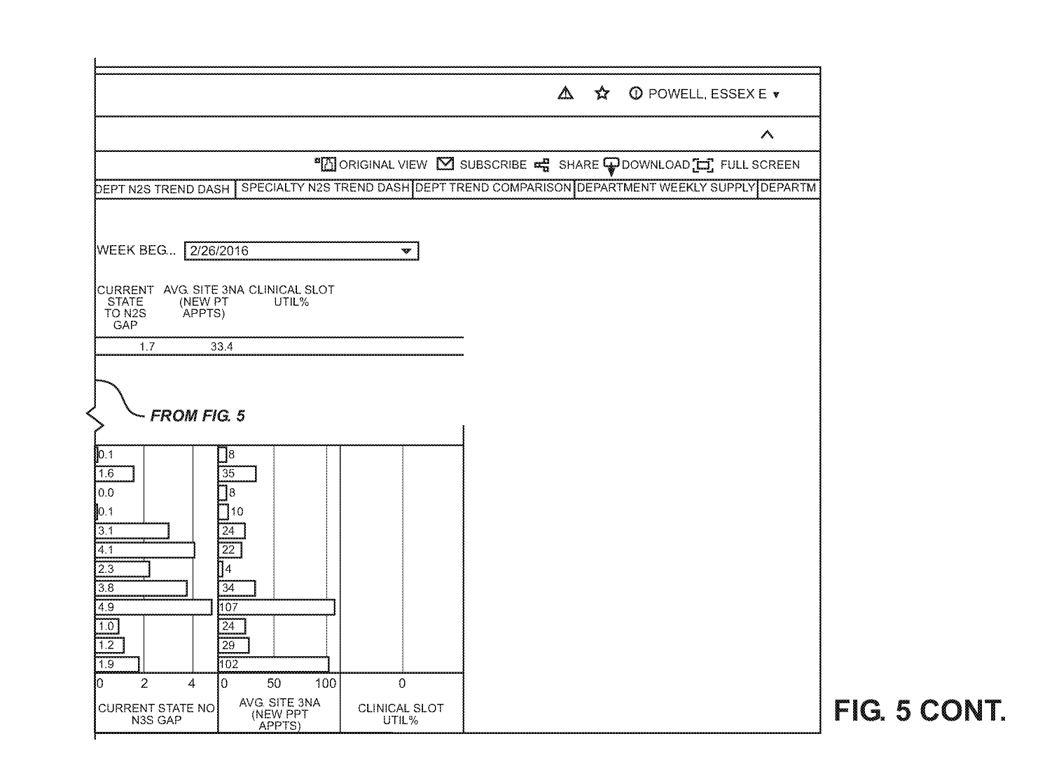

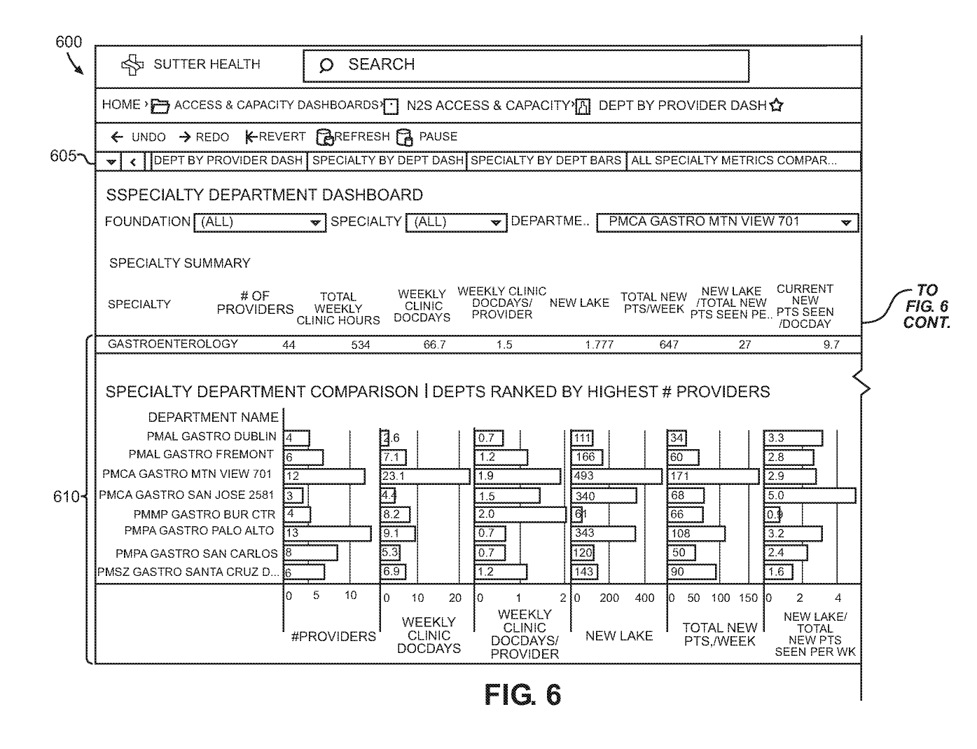

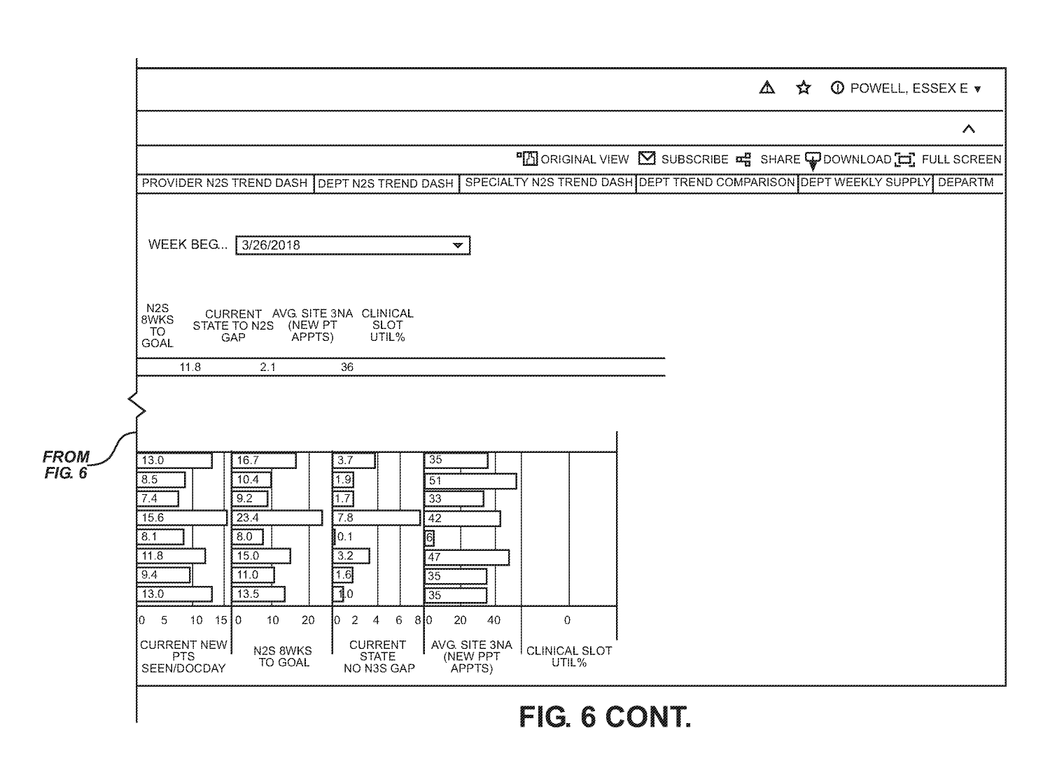

FIGS. 5-6 show various exemplary improved user interfaces 500 and 600, respectively, for enhanced healthcare patient access and treatment.

FIG. 5 shows a department by provider N2S user interface 500. Among other features, the N2S user interface includes numerous menu selections 505. As illustrated here, the relevant N2S components are shown for a particular department and for each of the providers in the department, as illustrated in frame 510.

FIG. 6 shows a specialty by department N2S user interface 600. Based on another menu selection (from menu selections 605), the relevant N2S components for a particular specialty in total is illustrated in frame 610, along with the relevant N2S components by department, ranked by the highest number of providers.

As illustrated in FIGS. 5 and 6, a computing device is configured to display on the screen a menu listing (such as listings 505 and 605, respectively) showing one or more applications, and additionally being configured to display on the screen an indicator (such as indicator 310 in FIG. 3) that can be reached directly from the menu, wherein the indicator displays a limited data set offered within the one or more applications.

In various exemplary embodiments, a launcher lists names and/or icons of several applications and selecting a name or icon causes a summary for that application to be opened, such as shown in FIG. 2, item 315 (FIG. 3), interface 400 (FIG. 4) and frames 510 and 610. Additionally, the kind of content of a summary for a given application may be selected using a process in which the device learns what data type is of interest to any given user. Alternatively, a user can define what data type is of interest to that user for the summary for an application. In some cases, a data type for a summary for a given application may vary with the environment of the device, such as the physical location of the device. In further cases, the data type for a summary for a given application may vary with the actions of a user.

The summary, in various exemplary embodiments, may be a frame which includes the name of one or more applications. The summary may further display a limited list of functions offered in the one or more applications. Additionally, some or all of the information may be displayed in a fashion that complies with Health Insurance Portability and Accountability Act. This feature may also be based on the physical location of the device.

The improved user interfaces for enhanced healthcare patient access and treatment as described and illustrated herein offer numerous advantages including improvements to the way information is displayed on user interfaces, particularly with regard to presenting a limited set of information that improves the functionality of the interface to the user. The various exemplary embodiments provide a specific, structured graphical user interface paired with a prescribed functionality directly related to the graphical user interface's structure that is addressed to and resolves a specifically identified problem in the prior state of the art.

The improved user interfaces for enhanced healthcare patient access and treatment as described and illustrated herein offer functional tools for enhancing provider supply on an individual level, group of providers level, or an entire organizational level. The user interfaces allows the estimation of the impact of the addition and subtraction of providers to an access ecosystem.

While various embodiments have been described above, it should be understood that they have been presented by way of example only, and not limitation. The descriptions are not intended to limit the scope of the technology to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the technology as defined by the appended claims and otherwise appreciated by one of ordinary skill in the art. The scope of the technology should, therefore, be determined not with reference to the above description, but instead should be determined with reference to the appended claims along with their full scope of equivalents.

What is claimed is:

1. A computing device comprising a display screen, the computing device being configured to dynamically display a specific, structured interactive graphical user interface paired with a prescribed functionality directly related to the interactive graphical user interface's structure.

2. The computing device of claim 1, further comprising the structure dynamically displaying a limited set of information to a user.

3. The computing device of claim 1, further comprising the prescribed functionality utilizing a N2S methodology.

4. The computing device of claim 3, further comprising the N2S methodology causing transformation of a message into a specifically dynamic visually perceptible graphical icon.

5. The computing device of claim 4, the dynamic visually perceptible graphical icon representing a condensed information set.

6. The computing device of claim 5, further comprising the condensed information set being dynamically displayed along with another condensed information set on a limited screen size.

7. The computing device of claim 6, further comprising the limited screen size being on a mobile computing device.

8. The computing device of claim 4, further comprising the transformation increasing a speed of the display.

9. The computing device of claim 4, further comprising the transformation increasing an amount of information dynamically displayed on a limited screen size.

10. A computing device comprising a display screen, the computing device being configured to display on the screen a menu listing one or more applications, and additionally being configured to display on the screen an application summary that can be reached directly from the menu, wherein the application summary displays a limited list of data offered within the one or more applications.

11. The computing device of claim 10, wherein the application summary is displayed while one or more other applications are not displayed.

12. A computing device comprising a display screen, the computing device being configured to display on the screen a menu listing one or more applications, and additionally being configured to display on the screen an indicator that can be reached directly from the menu, wherein the indicator displays a limited data set offered within the one or more applications.

13. The computing device of claim 12 in which an application launcher lists names or icons of several applications and selects a name or icon that causes a summary for that application to be opened.

14. The computing device of claim 12 in which a kind of content of a summary for a given application is selected using a process in which the device learns what data type is of interest to any given user.

15. The computing device of claim 12 in which a user can define what data type is of interest to that user for the summary for an application.

16. The computing device of claim 12 in which a data type for a summary for a given application varies with the environment of the device.

17. The computing device of claim 12 in which the data type for a summary for a given application varies with the actions of a user.

18. The computing device of claim 12 in which the summary is a frame which includes the name of one or more applications.

19. The computing device of claim 12 in which the summary further displays a limited list of functions offered in the one or more applications.

20. The computing device of claim 12, being a PC.

21. The computing device of claim 12, further comprising displaying some or all information in a fashion that complies with Health Insurance Portability and Accountability Act.

22. The computing device of claim 13, the icon representing a thumbs-up symbol with a numerical indicator between a thumb and index finger.

23. The computing device of claim 22, the numerical indicator representing a performance standard.

24. The computing device of claim 23, the performance standard based on N2S methodology.

25. The computing device of claim 16 in which a data type for a summary for a given application varies with the environment of the device and dynamically displays some or all information in a fashion that complies with Health Insurance Portability and Accountability Act as appropriate for the environment of the device.
